(19) United States
(12) Patent Application Publication
(10) Pub. No.: US 2014/0271890 A1
(43) Pub. Date: Sep. 18, 2014

(54) CONTROLLED-RELEASE PHARMACEUTICAL COMPOSITION

(71) Applicant: Thaer Ahmad, Upland, CA (US)

(72) Inventor: Thaer Ahmad, Upland, CA (US)

(21) Appl. No.: 13/826,871

(22) Filed: Mar. 14, 2013

(51) Int. Cl. A61K 9/24

(52) U.S. Cl. CPC A61K 9/209
USPC 424/494; 424/195.17; 424/490; 424/498; 424/497

(57) ABSTRACT

The present invention relates to a controlled-release pharmaceutical composition which, when administered in the evening, promotes rapid sleep onset and makes it easy to wake up in the morning in a refreshing way. The composition comprises: a first portion having sleep-inducing activity, which is able to be degraded and absorbed in vivo within 5 minutes to 1 hour after administration; and a second portion having cognition-enhancing activity, which is able to be released in vivo after 4 to 8 hours from the start of absorption of the first portion. The composition, when administered in the evening, promotes rapid sleep onset and makes it easy to wake up in the morning in a refreshing way.

### BACKGROUND OF THE INVENTION

1. Field of the Invention

The present invention relates to a controlled-release pharmaceutical composition which, when administered in the evening, promotes rapid sleep onset and makes it easy to wake up in the morning in a refreshing way.

2. Description of the Prior Art

Sleep problems are prevalent worldwide in humans (see Roth, T. et al., Sleep Med 6:487-95, 2005). Generally, sleep refers to a state in which the consciousness of the body is at rest with eyes closed. It is also a process of providing energy for physical activity, removing waste matter from the body, and recovering the body from fatigue. Thus, sleep is not only a process for energy provision, waste matter removal and fatigue recovery, but also a time in which growth hormones required for development are most actively secreted.

The human brain is an organ that controls all physiological and mental functions for life maintenance. In addition, the human brain needs rest to perform its greatest, and this rest is generally achieved by sleep. Sleep deprivation adversely affects health, and various recent studies show that sleep deprivation increases risk of diabetes, heart diseases, obesity and the like.

When describing sleep concerns, subjects usually complain of difficulty initiating sleep (DIS), of difficulty maintaining sleep (DMS), of awakening too early in the morning, or of a combination of these symptoms. However, a fourth complaint related to poor sleep has been described in the last several years as non-restorative sleep (NRS)—a feeling that the sleep episode has been un-refreshing or un-restoring—, or what the DSM-IV describes as light, restless, or poor quality sleep (see American Psychiatric Association, Diagnostic and Statistical Manual of Mental Disorders (4^{th} ed, Text Revision, 2000)). Complaints from subjects suffering from non-restorative sleep (NRS) include difficulty getting started in the morning, daytime fatigue, daytime sleepiness, general inability to function in the daytime, alertness problems, impaired mood, and poor work and academic performance. For those suffering from NRS, however, these complaints may not be the result of difficulties initiating or maintaining sleep. This was demonstrated in a multinational epidemiology study comprised of over 25,000 individuals, which showed that about 11% of the study population experienced NRS and that about 3% of the study population experienced NRS without the classic symptoms of DIS or DMS (see M. Ohayon, Arch Intern Med 165:35-41, 2005).

In addition, a certain number of hypnotic agents with varying modes and durations of action have been developed over the years. For example, a first category of hypnotic agents consists of those with a short duration of action. As used herein, the term “short-acting hypnotic agent” means a compound that acts mainly as a sleep inducer, i.e., a compound that acts on the time for entering into the sleep phase. For example, zolpidem is a short-acting hypnotic agent, which acts as a GABA-A receptor modulator. Zolpidem belongs to the class of imidazopyridines, and is administered orally in the form of an immediate-release tablet or in a galenical form allowing delayed release. Zolpidem acts quickly, is absorbed well and has a bioavailability of 70%. The mean dose, between 5 and 10 mg in a conventional formulation, induces a maximum plasmatic concentration that is reached between 0.5 and 3 hours, the half-life is short, with a mean value of 2.4 hours and a duration of action ranging up to 6 hours. Other examples of short-acting hypnotic agents are zaleplon, which belongs to the class of pyrazolopyrimidines, zopiclone and eszopiclone, which belong to the class of cyclopyrrolones, and also derivatives thereof.

Long-acting hypnotic agents have also been developed. As used herein, the term “long-acting hypnotic agent” means a compound that acts mainly on the quality and/or maintenance of sleep, especially the phases of deep sleep. Such a long-acting hypnotic agent is, for example, eplivanserin. Eplivanserin is a 5HT2A receptor inhibitor that acts without blocking dopamine. Eplivanserin and a preparation method thereof are especially described in EP-A-0 373 998. Eplivanserin is also absorbed well, with a bioavailability of 80%. The conventional dosage, between 1 and 10 mg, induces a maximum plasma concentration is reached between 2 and 6 hours, the half-life time being relatively long, with a mean value of 50 hours. Other long-acting hypnotic agents are, for example, gaboxadol and pregabaline, and also derivatives thereof. These hypnotic agents allow sleep disorders to be treated, especially insomnia.

However, whereas short-acting hypnotic agents act mainly on the entry into the sleep phase, long-acting hypnotic agents act rather on the phase of deep sleep. In addition, the hypnotic agents may, especially when they are administered at high doses, have a negative impact on the awake periods, in particular that following the taking of the medicament. Thus, it is still desirable to have an available composition that can induce or maintain repairing sleep even at a low dose.

In addition, humans may need to wake up from sleep within a suitable short time for their social activity. However, it is very difficult to ensure early awakening together with sufficient sleep.

### SUMMARY OF THE INVENTION

Accordingly, the present invention has been made in view of the problems occurring in the prior art, and it is an object of the present invention to provide a pharmaceutical composition which can exhibit the effect of inducing sleep within a short time after administration together with the effect of making it easy to wake up in a refreshing manner within a suitable time after sleep onset.

To achieve the above object, in accordance with one aspect of the present invention, there is provided a controlled-release pharmaceutical composition comprising: a first portion having sleep-inducing activity, which can be degraded and absorbed in vivo within 5 minutes to 1 hour after administration; and a second portion having cognition-enhancing activity, which can be released in vivo after 4 to 8 hours from the start of absorption of the first portion.

Other objects and advantages of the present invention will be understood from the following detailed description. Further, it will be readily understood that the objects and advantages of the present invention can be realized by the means as claims and combinations thereof.

### DETAILED DESCRIPTION OF THE INVENTION

Hereinafter, preferred embodiments of the present invention will be described in detail. Prior to description, it should be noted that the terms or words used in the specification and claims should not be construed as having common and dictionary meanings, but should be interpreted as having meanings and concepts corresponding to the technical sprit of the present invention based on the principle that the inventor can properly define the concepts of the terms in order to describe his/her invention in the best way. Thus, the embodiments described in the specification and the configurations shown in the drawings are simply the most preferable examples of the present invention and are not intended to illustrate all aspects of the spirit of the present invention. Accordingly, it should be understood that various equivalents and modifications that replace these embodiments are possible at the time of filing of the present application.

In accordance with one embodiment of the present invention, there is provided a controlled-release pharmaceutical composition comprising: a first portion having sleep-inducing activity; and a second portion having cognition-enhancing activity. The first portion having sleep-inducing activity can be degraded and absorbed in vivo within about 5 minutes to about 1 hour, preferably about 10 minutes to about 30 minutes, after administration, and the second portion having cognition-enhancing activity can be released in vivo after about 4-8 hours, preferably about 5-7 hours, from the start of absorption of the first portion. The first portion having sleep-inducing activity is absorbed in vivo within the above-described time after administration of the pharmaceutical composition to a subject to help the subject to initiate sleep in a comfortable and suitable manner. In addition, the second portion having cognition-enhancing activity helps wake up in the morning without unpleasant feeling or grogginess by improving cognitive function, after the above-described time from the start of absorption of the first portion, that is, a sound sleep for the above-described time.

Preferably, the first portion of the pharmaceutical composition according to the present invention may comprise melatonin and a serotonin precursor.

Melatonin (N-acetyl-5-methoxytryptamine) is a hormone that is produced naturally in the pineal gland of the brain, particularly during night hours. Melatonin is made by synthesis in most cases, but is also extracted from the brain in some cases. Melatonin that has the following chemical structure can control the sleep-wake cycle and is also used as a health functional food.

[chemical structure]

Melatonin is not secreted at birth, but the secretion thereof becomes more vigorous with age, starts to decrease before and after adolescence and decreases to a very small amount in senescence. In other words, it appears that aging occurs with a decrease in melatonin. Melatonin is synthesized from the essential amino acid tryptophan in the pineal gland that is a small tissue located in the center of the brain. Melatonin is structurally similar to tryptophan, and thus has a potent antioxidant activity that is about two times higher than vitamin E known as an antioxidant. Thus, it is very useful for anti-aging.

Melatonin that is used in the present invention may be natural or synthetic melatonin. When natural melatonin is used, it may be extracted from a material having a high content of melatonin, or a material having a high content of melatonin may also be used in the present invention. For example, oats known to containing a large amount of melatonin do not satisfy the daily need of melatonin, because the melatonin content is about 1.8×10⁻⁶ mg/kg. Thus, extracted and concentrated melatonin or synthesized melatonin should be used. In addition, melatonin may also be used in the present invention, but is less desirable because of the possibility of contamination with proteins that can cause viral or antibody reactions.

Serotonin (5-hydroxytryptamine (5-HT)) is a monoamine neurotransmitter. Serotonin that is biochemically derived from tryptophan is primarily found in the gastrointestinal tract, platelet and central nervous system of animals, including humans. Serotonin influences cerebral cortexes, autonomic nerves, muscles, senses and the like to solve human emotional problems such as depressive disorder, personality disorder, eating disorder, anxiety disorder and the like. Thus, serotonin is a contributor to feeling of happiness. Serotonin has the following chemical structure, and Precursors of serotonin include L-tryptophan, 5-hydroxytryptophan (5-HTP), and the like.

[chemical structure]

Preferably, the first portion in the pharmaceutical composition according to the present invention may comprise melatonin, 5-hydroxytryptophan (5-HTP), phenibut, L-theanine and gamma-aminoburyric acid (GABA).

In addition, the content of melatonin in the first portion of the pharmaceutical composition according to the present invention is about 0.5-1 part by weight, preferably about 0.6-0.8 parts by weight, based on 100 parts by weight of the first portion. If the content of melatonin is less than 0.5 parts by weight, it cannot provide the desired antioxidant function, and if the content of melatonin is more than 1 part by weight, it can initiate sleep in an unready state or can inhibit ovulation in a very small amount of women.

In addition, the administration of melatonin shows a hypnotic effect of enhancing the quality and time of sleep. It improves mood in some people. For younger people, administration of about 5 mg of melatonin can induce sleep. The time for the peak hypnotic effect varies, and the administration of melatonin at a daily dose of about 1-2 mg for about 3 weeks enhances the quality and time of sleep in insomniac old people. However, Jean-Louis et al. reported that the use of 6 mg of melatonin in 10 old people in a cross-over experiment did not significantly improve the quantity and quality of sleep. Thus, the hypnotic effect of melatonin is not entirely satisfactory. In fact, melatonin alleviates jet lag syndromes.

In addition, the content of 5-HTP in the pharmaceutical composition of the present invention is about 30-40 parts by weight, preferably about 33-35 parts by weight, based on 100 parts by weight of the first portion. The content of phenibut in the composition of the present invention is about 10-20 parts by weight, preferably about 14-18 parts by weight, based on 100 parts by weight of the first portion. The content of L-theanine in the composition of the present invention is about 10-20 parts by weight, preferably about 14-18 parts by weight, based on 100 parts by weight of the first portion. The content of GABA in the composition of the present invention is about 30-40 parts by weight, preferably about 33-35 parts by weight, based on 100 parts by weight of the first portion. In addition, the first portion of the composition according to the present invention may further comprise a physiologically acceptable excipient.

Meanwhile, the second portion of the pharmaceutical composition according to the present invention may comprise first to fourth blends. Specifically, the first blend is a combination for supplying/enhancing energy; the second blend is a combination showing cognitive activity; the third blend is a combination showing antioxidant activity; and the fourth blend is a combination for promoting digestion and absorption.

The first blend may comprise sulbutiamine, L-tyrosine ((2S)-2-amino-3-(4-hydroxyphenyl)propanoic acid), Synephrine HCL, dimethylglycine, NADH (a reduced form of nicotinamide adenine dinucleotide (NAD)), optionally caffeine.

Generally, by “psychostimulant” or “psychostimulant agent” is meant a compound which causes an increase in dopaminergic and noradrenergic neurotransmission. More particularly, psychostimulants stimulate the neurotransmitters responsible for controlling attention, motivation, alertness and concentration. Dopamine and adrenaline are the two major neurotransmitters responsible for controlling attention, motivation, alertness, concentration. Examples of psychostimulants include morphine, modafinil, methylphenidate, deanol derivatives (acti 5, cleregil, debrumyl, etc.), ketoglutarate, adrafinil, sulbutiamine and the like.

Among them, sulbutiamine that is a synthetic derivative of thiamine is a lipophilic molecule that crosses the blood-brain barrier more easily than thiamine. It increases the levels of thiamine and thiamine phosphate esters in the brain. In addition, it leads to an increased formation of thiamine triphosphate (TTP) that acts as a regulator of the synaptic transmission of many neurotransmission systems. Such sulbutiamine has the following chemical structure and is known as a major component that influences the human brain function and actively reduces the stress of the body.

[chemical structure]

L-tyrosine ((2S)-2-amino-3-(4-hydroxyphenyl)propanoic acid), epinephrine, norepinephrine and dopamine is important brain neurotransmitters. Such neurotransmitters have the following chemical structures and influence neuronal consensus and mood.

[chemical structure]

Synephrine HCl that is an amphetamine-like substance is an alkaloid compound that acts as a neuro-stimulant. It is extracted from some plants, particularly Citrus aurantium. It is generally used as a safer weight control drug than ephedrine, even though the use thereof is strictly restricted due to its health risk factor.

[chemical structure]

Dimethylglycine (DMG) is a derivative of amino acid glycine that has a chemical structure of (CH₃)₂NCH₂COOH. It is generally found in beans and the liver of the body, and is a metabolic byproduct of choline. It can be formed by removing one methyl group from trimethylglycine. Such dimethylglycine can improve oxygen utilization so as to be used as an athletic performance enhancer and can act as an antioxidant and an agent for improving the functions of the immune and cardiovascular systems. In addition, some studies suggest that dimethylglycine improves brain functions and mental acuity and may be used as an agent for treating autism, epilepsy or mitochondrial disease.

NADH that is a reduced form of nicotinamide adenine dinucleotide (NAD) is a coenzyme involved in the Krebs cycle and helps produce energy in the body. In addition, it has been used to alleviate jet lag syndromes and treat chronic fatigue syndromes.

The second blend in the pharmaceutical composition of the present invention may comprise picalomine, vinpocetine, methylcobalamin, beta-PEA (Beta-Phenylethylamine), B-6 (pyroxidine), B-2 (Riboflavin) and B-1 (Thiamine).

The third blend in the pharmaceutical composition of the present invention may comprise a pine bark extract, a grape seed extract, Spirulina, Quercetin (2-(3,4-dihydroxyphenyl)-3,5,7-trihydroxy-4H-chromen-4-one), and alpha lipoic acid.

The fourth blend in the pharmaceutical composition of the present invention may comprise bioperine (extracted from Piper nigrum), a licorice root extract, a fennel seed extract, a ginger root extract, and L-arginine base.

The first blend in the second portion may comprise, based on 100 parts by weight of the second portion, about 5-15 parts by (preferably about 7-12 parts by weight) of sulbutiamine, about 5-15 parts by weight (preferably about 7-12 parts by weight) of L-tyrosine, about 5-15 parts by weight (preferably about 7-12 parts by weight) of Synephrine HCl, about 5-15 parts by weight (preferably about 7-12 parts by weight) of dimethylglycine, 0.5-1.5 parts by weight (preferably about 0.7-1.2 parts by weight) of NADH, and the balance of a physiologically acceptable excipient.

The second blend in the second portion may comprise, based on 100 parts by weight of the second portion, about 2.5-7 parts by weight (preferably about 3.5-4 parts by weight) of picalomine, about 0.5-5 parts by weight (preferably about 1-3 parts by weight) of vinpocetine, about 0.1-0.5 parts by weight (preferably 0.15-0.3 parts by weight) of methylcobalamin, about 5-15 parts by weight (preferably about 7-12 parts by weight) of beta-PEA, about 5-15 parts by weight (preferably about 7-12 parts by weight) of B-6, about 0.5-1.5 parts by weight (preferably 0.7-1.2 parts by weight) of B-2, about 0.5-1.5 parts by weight (preferably 0.7-1.2 parts by weight) of B-1, and the balance of a physiologically acceptable excipient.

The third blend in the second portion may comprise, based on 100 parts by weight of the second portion, about 0.5-5 parts by weight (preferably about 1-3 parts by weight) of a pine bark extract, about 0.5-5 parts by weight (preferably about 1-3 parts by weight) of a grape seed extract, about 0.5-5 parts by weight (preferably about 1-3 parts by weight) of Spirulina, about 2.5-7 parts by weight (preferably about 3.5-4 parts by weight) of Spirulina, about 5-15 parts by weight (preferably about 7-12 parts by weight) of alpha lipoic acid, and the balance of a physiologically acceptable excipient.

The fourth blend in the second portion may comprise, based on 100 parts by weight of the second portion, about 0.5-1.5 parts by weight (preferably about 0.7-1.2 parts by weight) of bioperine, about 0.5-1.5 parts by weight (preferably 0.7-1.2 parts by weight) of a licorice root extract, about 0.5-1.5 parts by weight (preferably 0.7-1.2 parts by weight) of a fennel seed extract, about 1-5 parts by weight (preferably about 2-4 parts by weight) of a ginger root extract, about 5-15 parts by weight (preferably about 7-12 parts by weight) of L-arginine base, and the balance of a physiologically acceptable excipient.

In addition, the first blend may further comprise caffeine. Caffeine is a well-known substance that is used as a neuro-stimulant with alertness, euphoria and diuresis. Caffeine may be used in an amount of about 5-15 parts by weight, preferably about 7-12 parts by weight, based on 100 parts by weight of the second portion.

In addition, the pharmaceutical composition of the present invention may further comprise a coating agent for controlling the in vivo release of the second portion. The present invention may also provide a pharmaceutical composition comprising: a shell consisting of the above-described first portion; and a core consisting of the above-described second portion. In addition, the pharmaceutical composition of the present invention may further comprise, between the shell consisting of the first portion and the core consisting of the second portion, a coating agent for controlling the in vivo release of the core consisting of the second phase.

The coating agent that is used in the present invention may be one or a mixture of two or more selected from the group consisting of calcium carbonate, potassium carbonate, dicalcium phosphate, magnesium hydroxide, HPMC (hydroxylpropyl methylcellulose), pectin, sodium alginate, stearic acid, carnuba wax, magnesium stearate, colloidal silica, Methocell, Solafloc, a pH-resistant polymers, ethanol, polyvinyl pyrrolidone, polyethylene glycol, polysorbate, and a vanilla flavor coating.

The pharmaceutical composition of the present invention can be formulated in any per se known manner or analogous methods thereof available with pharmaceutically acceptable carriers used in any per se known manner. The carriers include any ordinary organic and inorganic carrier substances that are usable in formulating medicines. For example, employable are excipients, lubricants, binders, disintegrators, etc. for formulating solid preparations. If desired, further employable are other additives such as ordinary preservatives, antioxidants, colorants, sweeteners, adsorbents, wetting agents, etc.

The excipients include, for example, lactose, white sugar, D-mannitol, starch, corn starch, crystalline cellulose, light silicic anhydride, etc. The lubricants include, for example, magnesium stearate, calcium stearate, talc, colloidal silica, etc. The binders include, for example, crystalline cellulose, white sugar, D-mannitol, dextrin, hydroxypropyl cellulose, hydroxypropylmethyl cellulose, polyvinyl pyrrolidone, starch, sucrose, gelatin, methyl cellulose, carboxymethyl cellulose sodium, etc. The disintegrators include, for example, starch, carboxymethyl cellulose, carboxymethyl cellulose calcium, croscarmellose sodium, carboxymethyl starch sodium, L-hydroxypropyl cellulose, etc. The solvents include, for example, water for injections, alcohol, propylene glycol, macrogol, sesame oil, corn oil, olive oil, etc. The solubilizers include, for example, polyethylene glycol, propylene glycol, D-mannitol, benzyl benzoate, ethanol, trisaminomethane, cholesterol, triethanolamine, sodium carbonate, sodium citrate, etc. The suspending agents include, for example, surfactants such as stearyl triethanolamine, sodium lauryl sulfate, lauryl aminopropionic acid, lecithin, benzalkonium chloride, benzethonium chloride, glycerin monostearate, etc.; hydrophilic polymers such as polyvinyl alcohol, polyvinyl pyrrolidone, carboxymethyl cellulose sodium, methyl cellulose, hydroxymethyl cellulose, hydroxyethyl cellulose, hydroxypropyl cellulose, etc. The isotonizing agents include, for example, glucose, D-sorbitol, sodium chloride, glycerin, D-mannitol, etc. The buffers include, for example, liquid buffers of phosphates, acetates, carbonates, citrates, etc. The soothing agents include, for example, benzyl alcohol, etc. The preservatives include, for example, parahydroxybenzoates, chlorobutanol, benzyl alcohol, phenethyl alcohol, dehydroacetic acid, sorbic acid, etc. The antioxidants include, for example, sulfites, ascorbic acid, etc. The stabilizers for light include, for example, titanium oxide, etc.

The pharmaceutical composition of the present invention can be provided in various dosage forms, for example, as tablets (including sugar-coated tablets, film-coated tablets), powders, granules, capsules (including soft capsules), etc., in accordance with various methods known in the art.

Hereinafter, the present invention will be described in detail with reference to examples. However, the embodiments of the present invention may be modified into various other forms, and the scope of the present invention should not be interpreted as being restricted to the embodiments. The embodiments are provided to more completely explain the present invention to those skilled in the art.

### Example 1

A pharmaceutical tablet composition according to Example 1 comprises a first portion and a second portion as follows.

The first portion comprises 1 mg of melatonin, 50 mg of 5-HTP (5-hydroxytryptophan), 25 mg of phenibut, 25 mg of L-theanine and 50 mg of GABA (gamma-aminoburyric acid); and the second portion comprises first to fourth blends, wherein the first blend comprises 50 mg of sulbutiamine, 50 mg of L-tyrosine ((2S)-2-amino-3-(4-hydroxyphenyl)propanoic acid), 50 mg of Synephrine HCL, 50 mg of dimethylglycine, 50 mg of NADH and 50 mg of caffeine; the second blend comprises 25 mg of picalomine, 10 mg of vinpocetine, 1 mg of methylcobalamin, 50 mg of beta-PEA, 50 mg of B-6, 5 mg of B-2 and 5 mg of B-1 5 mg; the third blend comprises 10 mg of a pine bark extract, 10 mg of a grape seed extract, 10 mg of Spirulina, 25 mg of Quercitin, and 50 mg of alpha lipoic acid; and the fourth blend comprises 5 mg of bioperine (extracted from Piper nigrum), 5 mg of a licorice root extract, 5 mg of a fennel seed extract, 15 mg of a ginger root extract, and 50 mg of L-arginine base.

The above-described components of the second portion were blended in a rotary blender and pressed using a rotary tablet press, thereby forming the second portion as a core.

A vanilla flavor coating as a coating agent was dispersed in water using a vortex mixer for about 30-90 minutes, and the dispersed coating agent was coated onto the surface of the second portion and dried, thereby forming a coated second portion.

The above-described components of the first portion were mixed and dispersed in water using a vortex mixer for about 30-60 minutes, and the dispersion was coated onto the surface of the coated second portion and dried, thereby preparing a tablet.

Drug Test

(1) Test sample: the pharmaceutical composition of Example 1.

(2) Test period: administered before going to bed once a day for 1 month (30 days) or more.

(3) Administration method: one tablet together with water was administered to each subject.

(4) Test subjects:

Inclusion Criteria
\textcircled{1} Men and women aged over 20 years old
\textcircled{2} Persons who complained of difficulty sleeping and of difficulty awakening in the morning.

Exclusion Criteria
\textcircled{1} Persons determined to have no difficulty in sleeping and in awakening in the morning.
\textcircled{2} Pregnant women, women suspected of being pregnant, and nursing women.
\textcircled{3} Persons suffering from gastrointestinal diseases, liver diseases, heart diseases or severe pollinosis.
\textcircled{4} Persons who have been administered with a drug that influences sleep-inducing and awakening effects.

Investigation Items
\textcircled{1} Sleep investigation: sleep states (sleep onset time after drug administration, sleep patterns, etc.) according to age and sex were recorded.
\textcircled{2} Investigation of awakening in the morning: whether awakening within 4-8 hours after sleep onset occurred, feelings (refreshing or twilight state), etc., were recorded.

2. Test Results

When the pharmaceutical composition of Example 1 was administered to the subjects, the quality of sleep within 1 hour after administration was significantly improved, awakening in the morning was easy, and the subjects could return to normal in a refreshing and clear state after awakening. Thus, from the results of this drug test, it could be seen that administration of the pharmaceutical composition of Example 1 makes it easy to initiate sleep, keep a sound sleep, wake up in the morning, and return to normal in a fresh state after awakening.

As described above, the controlled-release pharmaceutical composition according to the present invention, when administered in the evening, promotes rapid sleep onset and makes it easy to wake up in the morning in a refreshing way.

What is claimed is:

1. A controlled-release pharmaceutical composition comprising: a first portion having sleep-inducing activity, which is able to be degraded and absorbed in vivo within 5 minutes to 1 hour after administration; and a second portion having cognition-enhancing activity, which is able to be released in vivo after 4 to 8 hours from the start of absorption of the first portion.

2. The composition of claim 1, wherein the first portion comprises melatonin and a serotonin precursor.

3. The composition of claim 2, wherein the first portion comprises melatonin 5-HTP (5-hydroxytryptophan), phenibut, L-theanine and GABA (gamma-aminoburyric acid).

4. The composition of claim 3, wherein the first portion comprises, based on 100 parts by weight of the first phase, 0.5-1 part by weight of melatonin, 30-40 parts by weight of 5-HTP, 10-20 parts by weight of phenibut, 10-20 parts by weight of L-theanine, 30-40 parts by weight of GABA, and a balance of a pharmacologically acceptable excipient.

5. The composition of claim 1, wherein the second portion comprises: a first blend comprising sulbutiamine, L-tyrosine ((2S)-2-amino-3-(4-hydroxyphenyl)propanoic acid), synephrine HCL, dimethylglycine, NADH (a reduced form of nicotinamide adenine dinucleotide (NAD)), optionally caffeine; a second blend comprising Picalomine, Vinpocetine, methylcobalamin, beta-PEA, B-6, B-2, and B-1; a third blend comprising a pine bark extract, a grape seed extract, Spirulina, Quercitin, and alpha lipoic acid; and a fourth blend comprising bioperine (extracted from Piper nigrum), a licorice root extract, a fennel seed extract, a ginger root extract, and L-arginine base.

6. The composition of claim 5, wherein the first blend in the second portion comprises, based on 100 parts by weight of the second portion, 5-15 parts by weight of sulbutiamine, 5-15 parts by weight of L-tyrosine, 5-15 parts by weight of synephrine HCL, 5-15 parts by weight of dimethylglycine, 0.5-1.5 parts by weight of NADH, and a balance of a pharmaceutically acceptable excipient; the second blend comprises, based on 100 parts by weight of the second portion, 2.5-7 parts by weight of Picalomine, 0.5-5 parts by weight of Vinpocetine, 0.1-0.5 parts by weight of methylcobalamin, 5-15 parts by weight of beta-PEA, 5-15 parts by weight of B-6, 0.5-1.5 parts by weight of B-2, 0.5-1.5 parts by weight of B-1, and a balance of a pharmaceutically acceptable excipient; the third blend comprises, based on 100 parts by weight of the second portion, 0.5-5 parts by weight of the pine bark extract, 0.5-5 parts by weight of the grape seed extract, 0.5-5 parts by weight of Spirulina, 2.5-7 parts by weight of Quercitin, 5-15 parts by weight of alpha lipoic acid, and a balance of a pharmaceutically acceptable excipient; and the fourth blend comprises, based on 100 parts by weight of the second portion, 0.5-1.5 parts by weight of bioperine, 0.5-1.5 parts by weight of the licorice root extract, 0.5-1.5 parts by weight of fennel seed extract, 1-5 parts by weight of the ginger root extract, 5-15 parts by weight of L-arginine base, and a balance of a pharmaceutically acceptable excipient.

7. The composition of claim 6, wherein the first blend further comprises, based on 100 parts by weight of the second portion, 5-15 parts by weight of caffeine.

8. The composition of claim 1, wherein the composition further comprises a coating agent for controlling in vivo release of the second portion.

9. The composition of claim 1, wherein the first portion constitutes a shell, and the second portion constitutes a core.

10. The composition of claim 9, wherein the composition further comprises, between the shell and the core, a coating agent for controlling in vivo release of the core.

11. The composition of claim 8, wherein the coating agent is one or a mixture of two or more selected from the group consisting of calcium carbonate, potassium carbonate, dicalcium phosphate, magnesium hydroxide, HPMC (hydroxylpropyl methylcellulose), pectin, sodium alginate, stearic acid, carnuba wax, magnesium stearate, colloidal silica, Methocell, Solafloc, a pH-resistant polymers, ethanol, polyvinyl pyrrolidone, polyethylene glycol, polysorbate, and a vanilla flavor coating.
